(19) United States
(12) Patent Application Publication
(10) Pub. No.: US 2012/0058938 A1
(43) Pub. Date: Mar. 8, 2012

(54) NUTRITIONAL COMPOSITION INDUCING A POSTPRANDIAL ENDOCRINE RESPONSE

(75) Inventors: Robert Johan Joseph Hageman, Wageningen (NL); Mirian Lansink, Houten (NL)

(73) Assignee: N.V. NUTRICIA, Zoetermeer (NL)

(21) Appl. No.: 13/255,477

(22) PCT Filed: Mar. 9, 2010
(86) PCT No.: PCT/NL2010/050120

§ 371 Date: Nov. 22, 2011

(30) Foreign Application Priority Data
Mar. 9, 2009 (NL) PCT/NL2009/050106

(51) Int. Cl. A61K 38/22; A61K 38/26; A61K 38/27; A61K 38/30; A61K 38/11; A61P 25/00; A61P 3/10; A61P 3/04; A61P 1/16; A61P 21/00; A61P 1/00; A61P 1/04; A61P 9/00; A61P 25/18; A61P 25/24; A61P 25/16; A61P 25/08; A61P 5/00; A61K 38/28

(52) U.S. Cl. 514/5.2; 514/6.7; 514/7.2; 514/12.3; 514/12.6; 514/11.4; 514/8.5; 514/11.6; 514/10.9; 514/10.7; 514/9.7; 514/17.7; 514/6.9; 514/4.8; 514/17.8; 514/17.9

(57) ABSTRACT

The invention concerns the use of a nutritional composition for the treatment of disorders which are associated with malfunctioning in the uptake and use of food-derived energy in the human body. In particular, the invention concerns the use of a nutritional composition for the treatment of a disorder, which is mediated by a postprandial endocrine or neurological response in a human body, wherein the nutritional composition comprises one or more of a specific protein fraction, a specific carbohydrate fraction and a specific nutritional fiber fraction.

### FIELD OF THE INVENTION

The invention concerns the use of a nutritional composition for the treatment of disorders which are associated with malfunctioning in the uptake and use of food-derived energy in the human body. More in particular, the present invention concerns the use of a nutritional composition for the treatment of a disorder, which is mediated by a postprandial endocrine or neurological response in a human body, wherein the nutritional composition according the invention comprises one or more of a specific protein fraction, a specific carbohydrate fraction and a specific nutritional fiber fraction.

### BACKGROUND OF THE INVENTION

Persons suffering from said malfunctioning in the uptake and use of food-derived energy in the body are, for example, diabetes patients, patients suffering from the metabolic syndrome and obese persons, persons suffering from eating disorders, infants or children at risk of developing such disorders, persons suffering from irritable bowel syndrome and elderly suffering from dysfunction of a nervous system.

The aforementioned malfunctioning becomes evident by an abnormal high concentration of glucose and a variety of lipids in blood in the fasting state or after consumption of a food, or by excessive lipid deposition in the adipose tissues, or by hypofunction or malfunction of body tissues.

Despite the wide availability or application of treatment protocols and prevention measures, the above-mentioned medical problems are huge problems in terms of prevalence, nature of disorder (duration or severity) and consequences for the direct environment of the patient and for society.

Many nutritional compositions have been proposed in the past which would contribute to the treatment of said conditions. Known technical features in such nutritional compositions, which would contribute to solving the problem, were: the inclusion of fiber, a decrease in the glucose content of the food, and the inclusion of proteins which would increase insulin release by the pancreas.

Insulin is an endogenous hormone which increases glucose uptake by mammalian cells such as skeletal muscle cells, liver cells and adipose cells, and in this way decreasing blood levels of glucose. Dependent on the target tissue, such absorbed glucose is used as energy source for cell functioning, as substrate for synthetic purposes, e.g. for ribose, or as storage agent for the conversion into glycogen or lipids.

Glucagon is another endogenous hormone involved in carbohydrate metabolism. Produced by the α-cells of the pancreas, it is released when the glucose level in the blood is low (hypoglycemia), causing the liver to convert stored glycogen into glucose and release it into the bloodstream. The action of glucagon is thus opposite to that of insulin, which instructs the body's cells to take in glucose from the blood. An injectable form of glucagon is a vital first aid in cases of severe hypoglycemia when a subject is unconscious or for other reasons cannot take glucose orally. It is generally believed that (postprandial) glucose concentrations should be decreased by increasing postprandial insulin concentrations and decreasing postprandial glucagon concentrations.

WO 2007/004883 pertains to a carbohydrate fraction and use thereof for a flat postprandial glucose response.

WO 2008/054193 discloses nutritional compositions which comprise indigestible oligosaccharides for the improvement of insulin resistance, prevention of postprandial glycaemic dip and/or decrease of the postprandial glucose response of a meal.

E P 1 800 675 discloses compositions comprising polyunsaturated fatty acids, proteins and manganese and/or molybdenum for improving membrane compositions. It allows efficient treatment of immune related disorders.

U.S. Pat. No. 6,706,697 discloses a diabetic nutrition and weight loss drink powder composition comprising a major amount of isolated soy protein, milk protein isolate and fructose.

Magnoni et al. “Long-term use of a diabetes-specific oral nutritional supplement results in a low-postprandial glucose response in diabetes patients” Diabetes Research and Clinical Practice, vol 80 (2008), 75-82 shows an effect of a high-MUFA high-fibre diabetes-specific oral nutritional supplement on postprandial glucose response. Hageman et al. “A specific blend of intact protein rich in aspartate has strong postprandial glucose attenuating properties in rats” J. Nutrition vol. 138 (2008) 1634-1640 report that specific aspartate-rich blends of intact soy protein and alpha-lactalbumin significantly improve postprandial glucose response.

Haberer et al. “Beneficial effects on glucose metabolism of chronic feeding of isomaltulose versus sucrose in rats” Ann Nutr Metab vol. 54 (2009) 75-82 study the effect of plasma glucose and insulin after solid diets of isomaltulose, sucrose or corn starch.

The aforementioned documents are silent on the effects on glucagon.

A nutritional composition has now been found which—contrary to common knowledge—apart from increasing insulin concentration in the blood, also increases glucagon release after intake, and which is able to significantly decrease postprandial glucose levels and improve body function in a person suffering from said malfunctioning in the uptake and use of food-derived energy in the body. The nutritional composition according to the invention is capable of maintaining a relatively constant weight ratio of glucagon to insulin after intake. In addition, the nutritional composition according to the invention is capable, after enteral administration of the nutritional composition according to the invention, of changing the release of a wide range of endocrine products and of inducing a neurological response. This endocrine or neurological response results in the observed beneficial effect in postprandial glucose response, energy supply as well as other specific benefits.

### SUMMARY DESCRIPTION OF THE INVENTION

In one aspect, the present invention is concerned with the use of a nutritional composition for the treatment of a disorder, which is mediated by a postprandial endocrine or neurological response in a human body, wherein the nutritional composition according the invention comprises one or more of a specific protein fraction, a specific carbohydrate fraction and a specific nutritional fiber fraction.

The specific protein fraction is characterized by the presence of a vegetable protein. The specific carbohydrate fraction is characterized by the presence of at least one of galactose and lactose, or by the presence of isomaltulose, and the nutritional fiber fraction is characterized by the presence of a specific fraction of soluble fiber, in particular oligomers of galactose and resistant dextrin.

More specifically, the present invention is concerned with the use of nutritional composition for the treatment of a disorder, which is mediated by a postprandial endocrine or neurological response response in a human body, comprising at least one of a protein fraction, a digestible carbohydrate fraction and a nutritional fiber fraction, wherein the protein fraction comprises at least 30 weight % of a vegetable protein, the digestible carbohydrate fraction comprises 15 to 70 weight % of at least one of galactose and lactose and/or 10 to 65 weight % of isomaltulose, and the nutritional fiber fraction comprises 60 to 92 weight % of soluble fiber.

In one embodiment, the aforementioned composition further comprises one or more selected from the group consisting of melatonine, carnosine, anserine, octanoic acid, choline, betaine, and functional equivalents thereof, more preferably at least carnosine, anserine and/or octanoic acid.

Preferably, the nutritional composition according to the invention comprises at least two of a protein fraction, a digestible carbohydrate fraction and a nutritional fiber fraction, more preferably a protein fraction, a digestible carbohydrate fraction and a nutritional fiber fraction. Most preferably, the nutritional composition comprises a protein fraction, a digestible carbohydrate fraction and a nutritional fiber fraction, wherein the protein fraction comprises at least 30 weight % of a vegetable protein, the digestible carbohydrate fraction comprises (i) 15 to 70 weight % of at least one of galactose and lactose and/or (ii) 10 to 65 weight % of isomaltulose, and the nutritional fiber fraction comprises 60 to 92 weight % of soluble fiber.

In a preferred embodiment, the invention pertains to a nutritional composition for use in the treatment of a disorder which is mediated by a postprandial endocrine response in a human body, said postprandial endocrine response involving the sequential or simultaneous release of insulin and glucagon after intake of said composition, said composition comprising a protein fraction, a digestible carbohydrate fraction and a nutritional fiber fraction, wherein the protein fraction comprises at least 30 weight % of a vegetable protein, the digestible carbohydrate fraction comprises (i) 15 to 70 weight % of at least one of galactose and lactose, and (ii) 10 to 65 weight % of isomaltulose, and the nutritional fiber fraction comprises 60 to 92 weight % of a soluble fiber.

Also, the invention pertains to the use of a nutritional composition having the characteristics as described in the preceding paragraph, in the manufacture of a preparation for the treatment of a disorder which is mediated by a postprandial endocrine response in a human body, said postprandial endocrine response involving the sequential or simultaneous release of insulin and glucagon after intake of said composition.

According to certain embodiments of the nutritional composition according to the invention, other components or sources comprising such ingredients may be included as well, such as the dipeptides carnosine (beta-alanyl-L-histidine, a dipeptide of the amino acids beta-alanine and histidine) and/or anserine (beta-alanyl-N-methylhistidine, a dipeptide of the amino acids beta-alanine and N-methylhistidine), melatonin, octanoic acid and choline.

Also the invention concerns a method for the treatment of a disorder, which is mediated by a postprandial endocrine or neurological response in a human, said method comprising administering to said human a nutritional composition comprising one or more of the specific protein fraction, the specific carbohydrate fraction and the specific nutritional fiber fraction as described herein.

In one embodiment, the invention concerns a method for the treatment of a disorder which is mediated by a postprandial endocrine in a human body, said postprandial endocrine response involving the sequential or simultaneous release of insulin and glucagon after intake of said composition, said composition comprising the specific protein fraction, the specific digestible carbohydrate fraction and the specific nutritional fiber fraction as described herein.

In other words the invention concerns the use of one or more of a specific protein fraction, a specific carbohydrate fraction and a specific nutritional fiber fraction for the manufacture of a nutritional composition for the treatment of a disorder, which is mediated by a postprandial endocrine or neurological response in a human.

The invention can also be worded as a nutritional composition comprising one or more of a specific protein fraction, a specific carbohydrate fraction and a specific nutritional fiber for use in the treatment of a disorder, which is mediated by a postprandial endocrine or neurological response in a human.

### DETAILED DESCRIPTION OF THE INVENTION

In the context of this application, the term “or” between two members should be interpreted as referring to each member individually and in combination. Hence, the phrase “selected from A or B” refers to the selection of A, the selection of B and the selection of both A and B.

In the context of this application, when referring to a range, the term “at least” also includes the starting point of the range. For example, an amount of “at least 95 weight %” means any amount equal to 95 weight % or above.

In the context of this application, enteral means orally or by tube.

In the context of this application, the % of total energy is also abbreviated as En %; En % is thus short for energy percentage and represents the relative amount that a constituent contributes to the total caloric value of the composition.

In the context of this application, the term “about” indicates that a certain deviation is allowed from a cited value, the magnitude thereof being determined by inter alia the accuracy of the determination method. Typically, such a deviation is 10%.

### Postprandial Endocrine Response and Neurological Response

The nutritional composition, after intake of appropriate amounts, is capable of modulating simultaneously or sequentially the release of endogenous gut hormones or of inducing a neurological response in a human body. These responses result in an optimal supply of components to body tissues in terms of the amount of components, which is available to peripheral tissues, per time unit, and the duration of this supply. These components include the amount of energy as present in the food.

The nutritional composition therefore does not, after intake, induce the maximum response for one individual gut hormone, but causes the sequential or simultaneous release of a combination of several gut hormones and the induction of a neurological response, which is beneficial in the treatment of the diseases and disorders, as mentioned hereafter. In particular, the nutritional composition is capable of generating a simultaneous release of insulin and glucagon by the endocrine tissues and cells, predominantly those present in the gastrointestinal tract, which release becomes evident when measuring the amount of these hormones in body tissues, like blood.

Additionally, administration of the nutritional composition may cause a broad range of neurological and endocrine effects, which have profound effects on energy use and fluxes of energy carriers, such as glucose, glycogen and lipids within the body. Administration of the nutritional composition according the invention results in a pattern of sequential or simultaneous release of several hormones and neurotransmitters from the tissues in the gastrointestinal tract or other tissues. Examples of such hormones include somatostatin, gastrin, glicentin, oxyntomodulin, pancreatic polypeptide (PP), reeelin, somatotropin, amylin, cholecystokinin (CCK), leptin, gastric inhibitory polypeptide/glucose-dependent insulinotropic peptide (GIP), glucagon like peptides (GLP-1 or GLP2), protein YY, IP1 and IP2, MPGF, human growth hormone, insulin like growth factor (IGF-1), tachykinins, resistin, adipsin, serotonin, melanocortins, corticotrophin releasing hormone, oxytocin, neuropeptides, orexins and histamine.

In a specific embodiment, the nutritional composition could also have a positive effect on the rate of catabolism of glucagon-like peptides and several other proteins, most likely by inhibiting the activity of dipeptidyl peptidase nr 4 (DPP-IV), the peptidase, which converts and deactivates the glucagon-like peptides and other currently known substrates of this peptidase. This induces a lengthening and increase of the activity of for example GLP-1 and several neuropeptides, chemokines and growth factors.

In a further embodiment of the invention, the nutritional composition may cause a decrease of the urinary excretion of glucose. Such excretion is a large problem in diabetic patients, because a large amount of glucose from the diet is lost for use by the body via this route. Hence, the nutritional composition according the invention may increase the amount of glucose utilization by the body tissues. The rate of urinary excretion can be measured using methods known in the art and be expressed as amount of glucose excreted by urine per mg creatinine or per 24 hour.

These effects may be achieved by interactions with various cells in the gastrointestinal tract, such as ECL-cells, G-cells, S-cells, P-cells, EC-cells, L-cells, enterochromaffin cells, neurons and glial cells in the enteric nervous system, and by interactions with cells in the pancreas, like the stem cells, the α- and β-cells, the D-cells and PP-cells and by interacting with brain cells, such as cells in the brain stem, the hippocampus, hypothalamus, cerebellum and pituitary gland.

The postprandial endocrine and neurological response can therefore be considered as a reaction of the human body to the oral administration of the nutritional composition according to the invention, wherein the reaction includes a sequential or simultaneous pattern of releases of hormones or signaling compounds or an activation of one or more nervous systems in the body.

The pluriform or multifactorial character of the nutritional composition creates a multitude and complex combination of effects which cause the net results as shown. Without being bound by theory, the way the nutritional composition according to the invention causes an effect can be characterized by inducing the release of a specific ratio of endocrine products, which can be measured in blood plasma after intake, or by inducing a neurological response, which can be measured by determining e.g. vagal activity (nervus vagus firing rate) or activating serotoninergic neurons in the enteral or brain tissues. In order to achieve a claimed effect, specific aspects of the total induction pattern of the endocrine or neurological response as caused by administration of the nutritional composition, are most important. Thus, for example for causing the effect on satiety the postprandial ratios of the concentrations of PYY, in particular the PYY3-36 nutritional composition thereof, reeelin and GIP seem very important, while for achieving the anabolic effects of the nutritional composition the induction of the release of specific weight ratios of hGH (human growth hormone), IGF-1, insulin and glucagon seem to be most relevant, and for understanding the effects on glucose metabolism of the nutritional composition, the release of specific weight ratios of insulin, glucagon, and optionally incretins like GLP and GIP or the neurological response e.g. those of serotoninergic neurons in intestine and brain seem to be most important.

According to one embodiment, the postprandial endocrine response is selected from at least 2, preferably at least 3, and most preferably at least 4 of the following group of responses: the release of insulin, glucagon, GLP-1, GLP-2, gastrin, amylin, PYY, GIP, reeelin, CCK, glicentin, hGH, IGF-1, oxytocin, vasopressin and melanocortins.

According to another embodiment, the postprandial endocrine response is the sequential or simultaneous release of insulin and glucagon.

According to another embodiment, the postprandial endocrine response is a neuro-endocrine response or involves an interaction with a nervous system or the pituitary gland.

According to a further embodiment, said interaction with a nervous system involves a stimulation of the nervus vagus, a change in the expression of serotonin receptors, an increased release of serotonine or an interaction with gaba-ergic neurons of the enteral nervous system, or an interaction with the hypothalamus, hippocampus, cerebellum, brain stem or pituitary gland.

In a preferred embodiment, after consumption of the nutritional composition, the ratio of glucagon (expressed in pMol/l) to insulin (expressed as mU/l) in blood plasma the ratio of glucagon (expressed in pMol/l) to insulin (expressed as mU/l) in blood plasma ranges between 0.7 to 1.3 pMol/mU, more preferably between 0.8 and 1.2 pMol/mU, most preferably between 0.85 and 1.1 pMol/mU.

In a preferred embodiment, after consumption of ready to serve unit of the nutritional composition, a glucagon concentration in blood plasma is achieved with a value above about 28 pMol/l (about 100 ng/l), and more between preferably between about 28 to about 45 pMol/l (about 100 to about 175 ng/l), in particular between about 31 to about 38 pMol/l (about 110 to about 130 ng/l).

When applying an immune-assay according to Gerich and Cryer, as disclosed in Cowett ed, Principles of Perinatal-Peonatal Metabolism, 2^{nd} Edition, Cowett R, ed. New York, Springer-Verlag, 1998, for determining the concentration of glucagon-type compounds, it is preferred that glucagon contributes at least 18%, more preferably 21 to 80%, most preferably 26 to 50%, in particular 29 to 42% of the total immune-reactivity which is measured in blood plasma.

Maintaining the balance between glucagon and insulin in a certain range has as major benefit that the dynamics of the flux of energy carriers, such as glucose, glycogen and lipids is increased in a human body. This occurs in a manner wherein sequentially or simultaneously a beneficial low postprandial glucose response and a beneficial effect on dyslipidaemias in the blood is achieved, as well as advantages in other body parts, such as the brain, the enteral nervous system, skeletal muscle (including smooth muscle in various organs and tissues), the liver, pancreas, intestinal tract, the adipose tissues, the kidney and the cardiovascular system.

Said dynamics allow increased flux of energy substrate in the body, thus resulting in an increased energy supply to tissues and cells, compared to nutritional compositions according to the state of the art, which aim to increase postprandial insulin release as much as possible and decrease the release of glucagon as much as possible, and do this by effecting postprandial ratios of glucagon/insulin below 0.7 pMol/IU.

It is important to note that the use of the nutritional composition according to the invention is also beneficial in conditions wherein the human is not hyper-insulinaemic, i.e. wherein the insulin concentrations after one night fasting are below 15 mU/l, preferably below 12 mU/l, most preferably below 10 mU/l, or even below 8 mU/1.

The endocrine response of the nutritional composition may activate a neurological reaction of the brain, in particular the hypothalamus. This reaction is called the neuro-endocrine response. The endocrine response may also induce a reaction of intestinal organs such as the pancreas or liver. The neurological response as induced by the nutritional composition includes a triggering of the enteral nervous system and includes an activation of the nervus vagus. In particular cholinergic and serotoninergic neurons are activated by the nutritional composition. In inflammatory conditions, also the activity of histaminergic receptors is modulated. The net result of responses affects for example satiety, eating patterns, digestion behavior, fate of administered or consumed food and the distribution of energy substrates such as glucose and lipids over the body.

### Nutritional Composition

The nutritional composition comprises several food components which are capable of inducing or inhibiting a release of gut hormones and influencing the neuro-endocrine system. However, it is important that the simultaneous intake of food components yields the required result, which is beneficial to the consumer.

The nutritional composition is characterized by the presence of at least one of a protein fraction, a digestible carbohydrate fraction and a nutritional fiber fraction, wherein the protein fraction comprises at least 30 weight % of a vegetable protein, the digestible carbohydrate fraction comprises 15 to 70 weight % of at least one of galactose and lactose or 10 to 65 weight % of isomaltulose, and the nutritional fiber fraction comprises 60 to 92 weight % of a soluble fiber.

Preferably, the nutritional composition according to the invention comprises at least two of a protein fraction, a digestible carbohydrate fraction and a nutritional fiber fraction, more preferably a protein fraction, a digestible carbohydrate fraction and a nutritional fiber fraction, wherein the protein fraction comprises at least 30 weight % of a vegetable protein, the digestible carbohydrate fraction comprises 15 to 70 weight % of at least one of galactose and lactose or 10 to 65 weight % of isomaltulose, and the nutritional fiber fraction comprises 60 to 92 weight % of a soluble fiber.

In order to be most effective, the nutritional composition is preferably administered in an amount of 4 to 40 g of dry matter, i.e. by administering the nutritional composition, 4 to 40 g of dry matter will be consumed, the dry matter of which comprises the active components. This dose of dry matter can be repeated several times, in order to feed a person, while achieving a desirable endocrine or neurological response.

In a preferred embodiment the nutritional composition is a liquid, which comprises a protein fraction, a digestible carbohydrate fraction and a fiber fraction as well as a lipid and mineral and vitamin fraction which has a volume between 120 and 240 ml, for example 125 ml, 200 ml, or 237 ml. The results as presented in Example 1, are obtained with such ready-to-serve nutritional composition having a volume of 200 ml.

During a period about 30 minutes to 120 minutes after the moment of administration of the nutritional composition, the endocrine or neurological response can be measured in blood. In other tissues this response may change with regard to the concentrations of the endocrine products and the duration of the concentration changes.

Optionally, other food components may be added for improved results in certain embodiments of the invention. For example, vitamins or minerals may be added, as well as carnosine or anserine, free arginine, choline or betaine and a lipid fraction, e.g. a lipid fraction comprising specific amounts of caprylic acid or docosahexaenoic acid. Preferably, said lipid fraction comprises docosahexaenoic acid (DHA) or a nutritional oil which comprises more than 2 g of DHA per 100 g of fatty acids. The total lipid composition in the ready to serve nutritional composition will thus have a DHA concentration of more than 0.3, preferably 0.38 to 40, more preferably 0.42 to 6 g per 100 g fatty acids in the nutritional composition.

### Protein Fraction

The protein fraction comprises a vegetal protein, i.e. a protein originating from plants, vegetables, fruits, like their leaves, stems, roots, seeds and other storage parts. Also algae belong to this group of vegetable proteins. Preferably, the protein is isolated from seeds, most preferably also from the seed's endosperm, of the plant. Examples of such a protein are proteins from soy bean, pea, beans, wheat, rice, lupin or from grains. Preferably, the protein is derived from soy. In particular, soy protein isolate is used. The vegetable proteins, especially the non-soluble proteins or non-whey proteins can be hydrolyzed or partially hydrolyzed in order to improve the solubility characteristics in liquid or semi-liquid nutritional compositions. When such proteins are hydrolyzed, they are preferably partially hydrolyzed, for example to an extent, wherein more than 40 weight % of the original protein has a degree of polymerization less than 4. This partial hydrolysis can be achieved by timely stopping the hydrolysis process. This can be achieved by denaturation of the hydrolyzing enzyme, for example by heat treatment, or by changing the hydrolysis composition, e.g. by adding inhibiting substances, or by changing the acidity.

Preferably, the vegetal protein according to the invention comprises more than 5, more preferably 6 to 10, and most preferably 6.5 to 9.5 weight % of L-arginine, in order to support the endocrine response of the intestinal tract. Alternatively, L-arginine, either in free form, as a salt, as a peptide or as a protein, may be included in the protein fraction, and ensures a more predictable release of insulin and glucagon in more patients, in order to induce the postprandial presence of insulin and glucagon in the weight ratio as claimed. Similar effects can be obtained by replacing this added L-arginine by ornithine or a salt thereof. Because added free L-arginine has a negative impact on taste and palatability, it is preferred to achieve the relatively high L-arginine content by selecting proper intact proteins or peptides comprising said L-arginine and having a molecular weight of more than 600 dalton, or by using ornithine or a salt thereof. A very suitable ornithine salt is ornithine ketoglutarate.

The vegetal protein from plants is included in an amount of more than 30 g, more preferably 36 to 90 g, most preferably 38 to 70 g per 100 g of the protein fraction.

Within the context of this application, the protein fraction is defined as the sum of all proteinaceous matter, and which is determined by summing up the amounts of proteins, peptides and amino acids as included in the nutritional composition.

In a preferred embodiment, the protein from the vegetable source is preferably combined with a protein of animal origin, such as proteins from mammalian milk, eggs and muscle or other tissues like blood. Preferably, the protein of animal origin is a milk protein, in particular a protein which is obtained by removing at least 65% by weight of the caseins of the protein. It is most preferred that the protein of animal origin is a protein which comprises aspartate in an amount which exceeds 10 g per 100 g amino acids in said protein of animal origin.

In a preferred embodiment, the protein of animal origin is a whey protein. As a source of whey protein to be used in the present invention, any commercially available whey protein source may be used, i.e. whey obtained by any process for the preparation of whey known in the art, as well as whey protein fractions prepared thereof, or the proteins that constitute the bulk of the whey proteins being β-lactoglobulin (about 65 weight %), α-lactalbumin (about 25 weight %) and serum albumin (about 8 weight %), such as liquid whey, or whey in powder form, such as whey protein isolate (WPI) or whey protein concentrate (WPC). Whey protein concentrate is rich in whey proteins, but also contains other components such as fat, lactose and glycomacroprotein (GMP), a caseine-related non-globular protein. Typically, whey protein concentrate is produced by membrane filtration. On the other hand, whey protein isolate consists primarily of whey proteins with minimal amounts of fat and lactose. Whey protein isolate usually requires a more rigorous separation process such as a combination of microfiltration and ultra-filtration or ion exchange chromatography. It is generally understood that a whey protein isolate refers to a mixture in which at least 90 weight % of the solids are whey proteins. A whey protein concentrate is understood as having a percentage of whey proteins between the initial amount in the by-product (about 12 weight %) and a whey protein isolate. In particular, sweet whey, obtained as a by-product in the manufacturing of cheese, acid whey, obtained as a by-product in the manufacturing of acid casein, native whey, obtained by milk microfiltration or rennet whey, obtained as a by-product in the manufacturing of rennet casein, may be used alone or in combination as source of globular whey proteins.

Furthermore, whey proteins may originate from all kinds of mammalian animal species, such as, for instance cows, sheep, goats, horses, buffalo's, and camels. Preferably, the whey protein is of bovine origin.

Preferably, the whey protein source is available as a powder, preferably the whey protein source is a WPC or WPI.

In another preferred embodiment, the whey is enriched in alpha-lactalbumin, i.e. additional alpha-lactalbumin is added to the whey.

In another preferred embodiment, the protein fraction essentially consists whey, optionally enriched in alpha-lactalbumin and soy in a whey:soy-weight ratio of 30:70 to 90:10, preferably in a 50:50 ratio.

The amount of total protein fraction should be more than 16% of the total energy (16 En %) which is provided by the nutritional composition after intake. This total amount can be calculated by using the Atwater constants for digestible carbohydrates and lipids and proteins (4, 9 and 4 kcal/g) and disregarding the amounts which might be provided from the other components in the nutritional composition of the invention. Preferably, the protein content is 18 to 80 En %, most preferably 19 to 70 En %. In one embodiment, the amount is 19.4 En %.

The protein and a complete nutritional composition comprising the protein are capable of significantly increasing glucagon release after intake and simultaneously maintain or even decrease plasma glucose concentrations.

### Digestible Carbohydrate Fraction

The nutritional nutritional composition according to the invention comprises a carbohydrate fraction. This fraction preferably contributes less than 50% of the total energy as provided by the total nutritional composition, more preferably 2 to 48 En %. In one embodiment, the amount of carbohydrate fraction is 46.6 En %. This carbohydrate fraction is preferably characterized by comprising 100% digestible carbohydrates. It is most preferred that the total carbohydrate fraction comprises a combination of digestible carbohydrates which are digested at different speeds. Preferably, the digestible carbohydrate fraction comprises more than 40 weight % of the sum of galactose or lactose and isomaltulose, more preferably 45 to 80 weight %. It is also preferred that the digestible carbohydrate fraction comprises 0.1 to 15 weight % of slowly digestible starch, i.e. starch which is digested between 0.5 and 2 hours in an in vitro assay under standardized conditions, for example resistant starch. It was found that such carbohydrate fraction contributes to the low postprandial glucose response and effects the postprandial neuro-endocrine response and release of gut hormones.

### Nutritional Fiber Fraction

The nutritional composition according the invention further comprises a nutritional fiber fraction. The fiber fraction comprises 60 to 92 weight % of a soluble fiber, preferably, 80 to 92 weight % of a soluble fiber. Examples of such soluble fibers are inulin, oligofructose, and hydrolyzed or partially hydrolyzed viscous fibers such as pectin, modified pectin, guar, carrageen, and others known in the art. More preferably, the fiber fraction comprises more than 32 weight % of galacto-oligosaccharides. In another embodiment, the fiber fraction comprises an oligofructose, in particular more than 80 weight % of an oligofructose, or less than 10 weight % of inulin. The oligofructose preferably comprises for more than 90 weight % compounds which comprise less than 6 monosaccharide units. The fiber fraction can also beneficially comprise 0 to 40 weight % of resistant dextrin. It was found that the fiber fraction according to the invention contributes to the low postprandial glucose response and allows the postprandial endocrine response and neurological response.

It was found that such fiber fraction contributes to the low postprandial glucose response and allows the postprandial neuro-endocrine response and release of gut hormones.

### Lipid Fraction

Optionally, the nutritional composition comprises a lipid fraction, which preferably contributes more than 31% of the total energy as provided by the nutritional composition. More preferably, the lipid composition contributes 32 to 80 En %, most preferably 33 to 45 En %. It is preferred that the lipid fraction comprises at least 0.3 g DHA per 100 g fatty acids in the nutritional composition. The included DHA facilitates the release of the intestinal hormones e.g. glucagon from α-cells (emiocytosis) The ratio of the weight of long chain polyunsaturated fatty acids (LC-PUFA's) of the omega 3 series to that of the omega 6 series is preferably in the range of 0.1 to 2, more preferably 0.15 to 1.6, most preferably 0.2 to 1.2, For calculation of omega 6 LCPUFAs, the amounts of C18 to C26 fatty acids having 2 or more unsaturated bonds are summed. For the calculation of the n-3, the amounts of C18 to C26 having 2 or more unsaturated bonds are summed.

### The Neurological and Endocrine Response

It is important to recognize that the combined use of food components exhibit the total—or net response after intake as disclosed. Optional fortification of the composition according to the invention with other disclosed ingredients may add specific modifications in the total response. The response, in particular the total—or net response can be characterized in many ways. It can be characterized by considering the insulin to glucagon response after intake, optionally in combination with the glucose response. However, it can also be characterized by considering a wide range of relevant biochemical parameters and—physiological interactions, or with the achieved clinical outcome, as described. The properties, i.e. the clinical efficacy of the nutritional composition can also be characterized by considering the relevant combinations of these parameters and interactions.

Preferably, the total endocrine response, as determined by the sequential or simultaneous action of nervous systems or modulation of the release of (a combination of) endocrine products, results in an improved handling of energy substrates by a human body, as becomes evident by higher transport fluxes of glucose and lipids between tissues in such body, compared to prior art products. This results in an excellent utilization of dietary carbohydrates and lipids by tissues and less lipid storage.

The inventors believe that plasma glucose concentrations are regulated by, for example, postprandial release of insulin and amylin by the pancreatic β-cells, glucagon by the pancreatic α-cells, by the release of incretins by various cells such as glucagon-like peptides (e.g. GLP-1 by the intestinal L-cells and GLP-2), GIP and CCK and by the effect of the food with the nervous system. The inventors also believe that the complete formula does allow increased glucagon release by a favorable postprandial release pattern of insulin and amylin, resulting in decreased postprandial insulin to amylin weight ratios or a lower amylin concentration, all in blood plasma, compared to state of the art formula. The inventors also believe that the interaction of the food with the nervous system causes a release of peptides like oxytocin, orexin, vasopressin, corticotropin releasing hormone from the brain and a regulation of the pancreas by the sympathetic and parasympathetic innervations of this organ, the direct activation of the nervus vagus or the activation of serotoninergic neurons in the brain.

The nutritional composition has a relevant clinical effect on satiety and food intake. The need for new food intake is decreased after consumption of the nutritional composition according the invention. This effect is quantified by measuring the voluntary food intake or alternatively by taking proper questionnaires. This makes the nutritional composition suitable for use in a weight management diet.

The neuro-endocrine response of the nutritional composition also delays gastric emptying or decreases the acid release in the stomach. This property makes the nutritional composition suitable for treatment of gastric ulcers and for combating gastric reflux, and stomach cancer.

If mitochondrial ATP-sensitive K+ channels are opened, e.g. as a result of PKC-activation, the intracellular Ca2+ concentrations go up. Also α-adrenergic receptor activation, as part of a neurological response, achieves this.

The nutritional composition according to the invention may exhibit an activating effect on one or more of the signaling of α1-adrenergic receptors, the serotonin 5HT2A and 2B receptors as located in the gastro-digestive tract (gastro-intestinal tract=GIT), and in particular in the smooth muscle cells thereof, as well as on the muscarin receptor M1, on the neurons of the GIT, and on the α1-receptors of the urinary tract. This allows proper contraction of the muscle cells and therefore proper function of the urinary sphincter and the propulsive force of the small intestine and colon (a consequence of proper gut motility). The improved gut motility is also explained by the PYY3-36 pattern which is released from the brain stem as a result of the intake of the nutritional composition.

The promoting effects of the nutritional composition on PKC-activation, in a preferred embodiment in brain cells, are further increased by including melatonin in the nutritional composition. The dose should exceed 0.2 g per daily dose and preferably 0.3 to 10, more preferably 0.4 to 1.4 g/daily dose. In a preferred embodiment the melatonin concentration is more than 0.1 g per 100 g of the nutritional composition.

The effects of the composition according to the invention also include a better regulation of body temperature. In particular the body temperature is maintained, preferably at a higher level in situations wherein food has not been consumed for more than 2 hours. The composition according to the invention restricts the physiological reaction of a human body to lower body temperature, when food intake was insufficient to maintain temperature homeostasis. This decrease in temperature is a general problem, but especially in women or elderly, for example in the evening. The problem manifests itself in a general feeling of uncomfortable cold, cold feet or cold hands, but also in a body temperature (core or peripheral), which is 0.5 to 3 degrees lower than normal.

The composition according to the invention as claimed prevents decrease of body temperature, or maintains body temperature for longer than 2 hours after administration of at least 200 kcal (840 kiloJoule), preferably 1.2 to 9 MJ, most preferably 1.5 to 5 MJ. This effect is probably achieved by the effect of the composition according to the invention on energy metabolism or the endocrine response. Without wanting to be bound by theory the inventors believe that the lower release of neuropeptide Y, and the increased release of orexins and oxytocin result in such improved temperature homeostasis. This also results in much better sleeping behavior, in particular shortening the time available to fall asleep or the continuous character of the sleep (undisturbed sleep time).

In order to increase or adapt the neurological response, optionally choline or betaine can be included in the composition according to the invention. For best results the amount of these components should exceed 0.24, preferably 0.4 to 13, more preferably 0.5 to 4 g per 100 g dry matter of the composition according to the invention. In a liquid nutritional composition according to the invention the concentration is preferably >0.04, preferably 0.06 to 0.4 g per 100 ml of the composition according to the invention. The choline can be administered as base or salt (e.g. chloride) or complex as well as in the form of a phospholipid or sphingolipids. Doses of the latter equivalents can be calculated by including equimolar amounts of the active component and correcting for molecular weight and purity of the ingredients. Betaine (also called betaine glycine) can be included in forms that are known in the art.

The nutritional composition also increases concentrations of glucagon-like peptides in brain and intestinal cells, probably due to increased expression of preproglucagon-mRNA, an improved processing of pre-proglucagon-mRNA to proglucagon or further to the peptides derived there from, or a better release of GLP-1 or GLP-2 from the synthesizing cells. The increase of the concentration of glucagon-like peptides in brain results in better cognitive and mental performance and better resistance against ischaemia-induced damage, like the damage which occurs during or after stroke and cerebrovascular events or accidents (CVA) and transient ischaemic accidents.

The higher GLP-1 release can be explained by higher proglucagon mRNA expression in e.g. colonocytes and results in increased energy expenditure in obese patients. The concomitant release of GLP-2 explains the trophic effects of the nutritional composition on the gut, in particular the colon.

In a specific embodiment of the invention, the nutritional composition is capable of inhibiting DPP-IV. The peptidase is inhibited, which results in a prolonged action and higher concentrations of GLP in a body tissue, but also of several growth factors, chemokines and neuropeptides. The effects is especially relevant when appropriate amounts of carnosine or anserine have been included in the nutritional composition. Each of these two dipeptides, as well as their functional analogs, are to be included, alone or in combination with each other, in an amount which exceeds 0.2 g per daily dose and preferably 0.3 to 10, more preferably 0.4 to 2.4 g/daily dose, in order to be effective. In a preferred embodiment the concentration of carnosine or anserine is more than 0.1 g, preferably 0.2 to 5 g per 100 g of the nutritional composition.

Functional analogs of these peptides are their salts, as known in food industry or pharmaceutical industry, and their esters with fatty acids, or organic acids like acetic acid or butyric acids and their ethers with alcohols like ethanol or n-butanol. Effective amounts of these analogs can be calculated by starting with the recommended molar amount of the effective compound (carnosine, anserine) and correcting for the molecular weight of the analog, as known in the art.

The effect of carnosine and or anserine and their functional analogscan also be achieved without including the protein, fibres or carbohydrates according the invention. In this embodiment, the nutritional composition can comprise the active components, carnosine or anserine in combination with a carrier, which is not a complete food matrix comprising a protein—a carbohydrate—or a fiber fraction. However, preferably the latter composition according to the invention comprises also an effective amount of octanoic acid (caprylic acid) or functional derivatives thereof, such as phospholipids, sphingolipids or triglycerides, which are enriched in octanoic acid or salts, ethers or esters of octanoic acid. Examples of suitable compounds are ingredients rich in medium chain triglycerides (MCT), especially those MCT which comprise more than 40 weight % of octanoic acid, and ethyl-esters of octanoic acid. The preferred dose of octanoic acid to be administered is more than 2.1 g, more preferably 2.7 to 14, most preferably 3.1 to 11 g per daily dose of the composition according to the invention. The dose of octanoic acid equivalents can be calculated by starting with the claimed amount of octanoic acid and correcting for the molecular weight of the equivalent compound, as known in the art. The weight ratio of [carnosine+anserine] to octanoic acid must preferably be in the range of about 0.1 to about 1.

Hence, in a specific embodiment of the invention, active components selected from carnosine or anserine or their functional; equivalents, and optionally one of octanoic acid or its functional equivalents, docosahexaenoic acid or its equivalents (e.g. triglycerides, phospholipids or sphingolipids comprising more than 2 g DHA per 100 g fatty acid in this ingredient), and choline or betaine or their functional equivalents is used for modulating an endocrine response or neurological response.

Such composition according to the invention can beneficially take the form of a premix for use in the manufacture of nutritional compositions to induce such endocrine or neurologic responses or be used for enteral administration as such. Useful composition are therefore emulsions or suspensions, which comprise per 100 ml 1 to 20 g carnosine or anserine equivalents, 2 to 20 g sphingolipids, lysolecithin or phospholipids which comprise choline, octanoic acid or docosahexaenoic acid, and 1 to 15 g choline or betaine, and optionally 0.4 to 10 g of a marine oil, the remainder being a suitable solvent like water or a mixture of water with an organic solvent like glycerol.

Such composition can also be a solid having less than 18 weight % moisture and comprising the above components in the amounts as given per 100 g dry matter: 2 to 40 g carnosine/anserine equivalents, 4 to 40 g sphingolipids or phospholipids or lyso phosphatides, and 2 to 30 g choline and or betaine or their equivalents and optionally 0.8 to 40 g marine oil. These active components can be mixed with digestible carbohydrates, a protein fraction, fiber other components or flavorings, vitamins, minerals or trace elements in order to prepare a palatable product. The sum of all components must obviously be 100%.

Obviously best results are obtained if the carrier comprises the food components as disclosed.

In a different embodiment of the invention the product is a liquid ready-to-use composition, comprising 18-20 En % protein, 30-47 En % carbohydrates and 31-52 En % lipids, and further comprising per 100 ml:
4.5 to 5.2 g protein equivalents (Kjeldahl nitrogen times 6.25), whereby the protein comprises 40 to 60 weight % soy protein isolate and 40 to 60 whey protein;
11.4 to 12 g digestible carbohydrates, wherein these carbohydrates comprise 3.2 to 4 g lactose, 4 to 4.9 g isomaltulose, 0.1 to 0.7 g glucose and 2.4 to 3.5 g polysaccharides, as are present in commercially available resistant dextrin or resistant starch or tapioca starch;
3.3 to 4.3 g lipids of which 0.08 to 2 g is marine oil and the remainder originates from canola oil and sunflower oil;
1.6 to 2.4 g fiber of which 0.2 to 0.5 is insoluble and 1.1 to 2.2 is soluble, the fiber comprising cellulose;
35-140 mg choline;
vitamins, trace elements and minerals are included in amounts which fall within the current regulations for “food for special medical purposes”.

The endocrine response induced by the product also includes a lower gene expression of resistin and adipsin in white adipose tissue and lower pituitary growth hormone-mRNA, compared to liquid nutritional products having an energy density above 1 kcal/ml and comprising casein, a digestible carbohydrate fraction with more than 60 weight % of carbohydrates which have a glycemic index (GI) above 80 and more than 30 En % of lipids. Preferably the product therefore comprises less than 40 weight % casein, less than 60 weight % carbohydrates having a glycemic index (against glucose) above 80, while comprising >30 En % of lipids and having an energy density of more than 1 kcal/ml.

In a specific embodiment, the nutritional composition is effective in the treatment of a disorder/disease selected from the group of diabetes type II, diabetes mellitus, diabetes type I, metabolic syndrome, obesity (BMI>28), diabetes as a result from the use of atypical antipsychotics, hepatosteatosis, excessive food- or energy consumption, abnormal urinary losses of glucose, muscle atrophy, lipid accumulation in muscle tissue, neurological disorders, irritable bowel syndrome, gut cramps, stomach disorders, like gastric reflux, excessive acid production in the stomach, e.g. as a result of stomatitis, cardiovascular problems, undesired weight loss, insufficient body-temperature homeostasis, sleeping problems and DPP IV-mediated disorders. DPP-IV mediated disorders include hyperglycemia and insufficient repair of brain after brain trauma.

In a further embodiment, the nutritional composition is effective in the treatment of neurological disorders selected from the group of age-related cognitive impairment, dementia, Alzheimer's, confusion, pathological thought disorders, schizophrenia, symptoms of schizophrenia, psychosis, affect disorders, depression, stroke, amyloid lateral syndrome (ALS), white matter abnormalities, nerve injury including spinal chord injury, multiple sclerosis (MS), cerebrovascular accidents (CVA), transient ischaemic accident (TIA), Parkinson's Disease (PD) and epilepsy.

### Induction of Fluxes of Energy Substrates in the Body

The nutritional composition ensures a better energy supply of peripheral tissues in the body compared to prior art nutritional compositions. This is due to a different form of metabolism or a higher flux of energy carriers from storage tissues to peripheral tissues, like muscle and brain. The latter flux is from glycogen or storage lipids to non-storage forms of energy. The nutritional composition according the invention has lower lipogenesis properties compared to prior art formulae.

Though the nutritional composition is not ketogenic in the meaning that the amounts of ketones, in particular the sum of acetone, β-hydroxy butyric acid and aceto-acetate, in blood plasma are increased considerably, the nutritional composition creates a condition in the body, wherein ketone bodies can be generated and used as energy substrate by tissues, which are in need of such ketone bodies. Examples of such tissues or cells, which benefit from additional ketone-body formation are those, which experience deficiencies in energy supply, e.g. those which are provided with insufficient blood supply, or those which have developed a glucose intolerance or insulin resistance, or persons, which are incapable of sufficient use of absorbed glucose due to metabolic restrictions. An example of the latter group of persons is the group of persons suffering from specific inherited metabolic diseases as known in the art, or epileptics.

In this way the nutritional composition is superior against those nutritional compositions which generate extremely high insulin/glucagon ratios. Because the neuro-endocrine response changes with age, as do the requirements, the dose of the active ingredients will change somewhat.

During aging changes occur in expression, localization, sensitivity of receptors, and in the release patterns of gut hormones and the neurological response as a result of feeding. This results in a changed digestion pattern, changed eating preferences and behavior, and “insulin resistance”. Insulin resistance reflects a situation wherein insulin which is released as a result of food intake is insufficiently effective to ensure proper glucose intake. This is observed as relatively high glucose concentrations after food intake. The inventors believe that in such situation the total endocrine and neurological response of the product ensures proper glucose absorption by most human cells or tissues, though insulin concentrations are not high. The plasma concentrations of glucose reflect the high fluxes of glucose in stead of an inability of cells and tissues to absorb glucose.

The neuro-endocrine response resulting from administering the nutritional composition, for example the relatively high glucagon/insulin response, optionally in combination with the growth hormone/IGF-1 response of the nutritional composition, allows lipolysis, e.g. lipolysis of adipose tissues, and the use of lipids by peripheral tissues, for example as energy substrate. This is measured as increased β-oxidation of the lipids.

These effects not only increase lean body mass, but also improve energy supply to tissues as the brain or enteral nervous system. This results in a better functioning of the nervous systems.

In particular this results in better treatment of epilepsy, an improved recovery after ischaemic events in nervous systems, like during stroke and cerebral-vascular accidents of permanent (CVA) or transient (TIA) character, less psychotic behavior and better cognitive functioning.

The lipolysis effect also contributes to improved nerve repair after damage due to lesions or trauma and leads to improved myelination, the latter being of special interest to patients suffering from white matter lesions, such as schizophrenics and persons suffering from multiple sclerosis and amyloid lateral syndrome.

The nutritional composition achieves also a relevant change in processing of lipoproteins. In particular it leads to a longer lasting and significant increase in the ratios of the weight of high density lipoproteins (HDL) to low density lipoproteins (LDL) in blood. This compensates for temporarily potentially disadvantageous increases in the plasma concentrations of triglycerides, which may result from increased lipolysis. This results in an improved prognosis for developing cardiovascular events and cerebrovascular events. These effects can be obtained with a product which comprises a protein fraction which consists of at least 20 weight % of animal protein and up to 80 weight % of protein of plant origin. Also very little caffeine or similar xanthines were present in the product; the amount of caffeine was less than 0.5 weight % or below 10 mg per ready to use serving.

In diabetic patients or persons suffering from the metabolic syndrome or elderly persons the increases in HDL/LDL ratio will start after at least 2 weeks of daily consumption of the product and will last for at least several days when the administration is suddenly stopped. The test persons appreciated very much the liquid formula, because of her good palatability and convenience of use. The liquid formula could be manufactured as a homogeneous suspension. The liquid formula could be made shelf stable for at least 1 year, by sterilization. The stabilizing system in this product comprised less than 50 weight % chitosan.

### DESCRIPTION OF THE FIGURES

FIG. 1—Twenty-four hour glucose concentrations (A), and mean glucose (B) during bolus feeding (5 times, 3 h between) with a diabetes-specific and standard formula. Plasma glucose concentrations were determined using CGMS. Bolus feeding was started at 8.00 a.m. (±1 h), corresponding to t=0 h. Daytime is defined as start time of first bolus until 3 h after start of the last bolus. Night time is defined as 3 h after start time of the last bolus until 24 h after start time of the first bolus. Values are means±SEM. *p<0.05.

FIG. 2—Peak plasma glucose concentration after each bolus and the mean peak plasma glucose concentration, determined during bolus feeding (5 times) using GCMS. Values are means±SEM. *p<0.05.

FIG. 3—Insulin (A) and glucagon (B) concentrations over 12 h during administration of the diabetes-specific formula and the standard formula. Values are means±SEM. *Significantly different with a significance level of 0.007 (Bonferoni adjustment).

FIG. 4—Postprandial reponses of insulin (Top) and glucagon (Bottom) of a nutritional product comprising a protein fraction and a digestible carbohydrate fraction, wherein the protein fraction comprises 50% soy protein+50% whey protein (filled symbols) or casein (open squares). The glucose response did not differ significantly.

### EXPERIMENTAL

### Experiment 1

### Materials and Methods

### Subjects

Patients were eligible to participate if they were ambulant type 2 diabetic patients according to Word Health Organisation criteria for more than 6 months. Other inclusion criteria were: (a) male: age >18 or post-menopausal females; (b) 5.5%≦HbA1c≦9.0%; (c) 18.0≦BMI≦35.0 (d) functioning gastrointestinal tract, eligible for tube feeding via a nasogastric tube; (e) on a stable and controlled anti-diabetic regime for at least 2 months; regimes were expected to remain stable throughout the duration of the study or not being on anti-diabetic medication; (f) if lipid lowering drugs were used, their use should be stable and controlled for at least 2 months and expected to remain stable throughout the duration of the study; (g) willingness to comply with the study protocol, including:
use of standard evening meal the day prior to the assessments
refrain form alcohol consumption (24 h) and intense physical activities (48 h) prior to, and during the assessments
not changing dietary and smoking habits for the duration of the study (except for the evening meal provided prior to every visit).

Exclusion criteria were (a) any gastrointestinal disease that interferes with bowel function and nutritional intake (i.e. diabetes related constipation or diarrhoea secondary to neuropathy, diarrhoea due to chronic inflammatory bowel disease, gastroparesis, gastrectomy) (b) significant heart (New York Heart Association class IV), hepatic (transaminase levels greater than 3 times normal) or renal disease (requiring dialysis) (c) major infections (requiring antibiotics) within 3 weeks before study entry (d) concomitant therapy with acarbose, meglitinides or insulin (e) concomitant therapy with systemic glucocorticoids or within 2 weeks prior to study entry (f) galactosaemia (g) patients requiring a fibre-free diet (h) alcohol abuse (i) investigator's uncertainty about the willingness or ability of the patient to comply with the protocol requirements (j) participation in other trials within 4 weeks of study entry.

The study protocol was approved by the local Ethics Committee and was performed in accordance with the principles relating to the Declaration of Helsinki. All participants gave written informed consent prior to study screening.

### Study Design

The study was designed as a randomized, controlled, double-blind, cross-over study and was performed at the Clinical Research Centre (CRCN) (Nijmegen, The Netherlands). Subjects were randomly assigned to one of two groups by a computer randomization program. Group I first received the diabetes-specific formula and next the standard formula and group II received the formulas in opposite order.

TABLE 1
Ingredients in the tested formulas

| | | Diabetes-specific | Standard |
|---|---|---|---|
| | | formula | formula |
| Ingredient | Unit | (per 100 ml) | (per 100 ml) |
| Energy | Kcal (KJ) | 100 (420) | 100 (420) |
| Protein | g/En % | 4.9/19 | 3.8/15 |
| whey protein | | ν | — |
| soy protein | | ν | — |
| casein protein | | — | ν |
| Carbohydrate | g/En % | 11.6/47 | 13.6/55 |
| isomaltulose | | ν | — |
| galactose & glucose | | ν | — |
| oligo- & polysaccharides | | — | ν |
| polysaccharides | | ν | — |
| slowly digestible starch | | ν | — |
| other | | ν | ν |
| Fat | g/En % | 3.8/34 | 3.4/30 |
| Saturated fatty acids | | ν | ν |
| MCT | | — | ν |
| Monounsaturated fatty | | ν | ν |
| acids | | | |
| Polyunsaturated fatty acids | | ν | ν |
| Fiber | g | 2.0 | 1.4 |
| Soluble | | ν | ν |
| Insoluble | | ν | ν |

The diabetes-specific formula (Danone Medical Nutrition Division, The Netherlands) was a newly developed formula (1 kcal/ml) based on low GI or slowly digestible carbohydrates (CHO) (47 En % CHO, 19 En % protein, 34 En % fat, 2 g fibre/100 ml). The standard formula was an isocaloric, commercially available fibre-containing formula (55 En % CHO, 15 En % protein, 30 En % fat, 1.4 g fibre/100 ml). Table 1 shows the characteristics of the two formulas. Subjects received 100% of their total daily energy requirements through both formulas. The total daily energy requirements were calculated using Harris-Benedict's equation (http://www-users.med.cornell.edu/˜spon/picu/calc/beecalc.htm with stress factor none and activity factor bed rest).

The subjects visited the research centre twice, with at least 4 but no more than 10 days in between. In order to avoid physical exertion prior to the measurements, all subjects stayed overnight at the clinic before the day of the 24 h glucose profile assessment. The evening before the assessments, subjects ate a standard meal and the Continuous Glucose Monitor System (CGMS, Medtronicl) was applied. Subjects continued the use of their oral anti-diabetic medication (if applicable) during the assessments. On the morning of the assessment, after an overnight fast of 10 h, a venous canula was placed in the forearm or dorsal vein and a nasogastric tube was inserted. Fifteen minutes after insertion of the tube, a fasting blood sample was taken whereafter recording of the glucose levels with the CGMS during 24 h was initiated. At 8.00 a.m. [symbol] 1 h, the first bolus of tube feed was given. Subjects received in total 100% of their total daily energy requirement as bolus feeding (equivalently distributed over five boluses) with 3 h in between. Thus, 12 h after the start of the first bolus feeding, the last bolus was given. Blood samples for determination of insulin and glucagon were collected just before start of a bolus and 1 and 2 h after the bolus. After the last bolus administration, no blood samples were collected, except for one blood sample at the end of the 24 h glucose monitoring. At several time points during the 24 h glucose monitoring, capillary blood glucose finger prick tests (Accu-Chek1 glucose meter) were performed to correlate to glucose measurements by GCMS. Tolerance to the formulas was determined with a questionnaire using a 7-point scale at various time points during the 24 h assessment. Assessed items were burping, bloating, stomach ache, abdominal cramping, nausea, flatulence, satiety, and well being. At the end of the 24 h assessment patients were also asked about the presence of diarrhoea and vomiting. Furthermore, adverse events (AEs and serious adverse events (SAEs)) were recorded during the study.

### Laboratory Methods

Body weight was measured to the nearest 0.1 kg using a weighing scale without wearing shoes or heavy clothing. Standing height was measured. Blood pressure was measured using standard equipment of the study centre. Analyses of blood samples were performed with commercially available equipment. Serum insulin concentrations were determined with an immuno chemoluminescense immunoassay (Immulite 2500, Siemens Medical Solutions Diagnostics, Munich, Germany). The determination of glucagon was performed with a radioimmuno assay (Siemens Medical Solutions Diagnostics, Munich, Germany). HbAlc was determined by high performance liquid chromatography (Menarini, Florence, Italy). The positive incremental areas under the curve (iAUC) were calculated for plasma glucose, according to the trapezoidal method [17].

### Statistical Analysis

Data of the intention to treat population are presented in this paper. The 24 h glucose profile was assessed by determination of mean glucose, incremental area under the curve (the CGMS glucose value, which was measured 5 min before start of the first bolus, was used as baseline), percentage of time above 10 mmol/L (hyperglycaemic periods) and percentage of time beneath 3.9 mmol/L (hypoglycaemic periods). These parameters were determined over 24 h, at daytime (start of the first bolus until 3 h after start of the last bolus) and at night time (3 h after start of the last bolus until 24 h after start of the first bolus). Delta peak glucose concentrations were calculated by subtracting fasting glucose concentration from peak glucose concentrations. Values are expressed as mean [symbol] SEM or as absolute number and percentages of subjects. The cross-over analysis consisted of three two sample t-tests testing treatment, period and carry-over effects, according to the methods described by Altman [18]. Efficacy and tolerance parameters were compared between the two treatments, periods and randomization groups. Categorical data (e.g. adverse events, early terminations) were compared between the two treatments, periods and randomization groups using Fisher's exact test. If normality assumptions (Shapiro-Wilk test) were not met and the p-value of the treatment effect of the parameter was between 0.01 and 0.10, the non-parametric Mann-Whitney test was used. Reproducibility of glucose measurements was estimated by calculating the regression coefficient of the paired data points of the CGMS readings and the capillary blood finger prick tests. p-Values were two-tailed and those <0.05 were considered statistically significant. To adjust for multiple comparisons of all separate time points of the secondary parameters a Bonferroni adjustment with an overall alpha of 0.10 was used. Analyses were performed using SAS, version 9.1.2 for Windows, Cary N.C.: SAS Institute Inc.

TABLE 2
Subject characteristics at baseline; ITT population

| | | Total |
|---|---|---|
| | | group |
| | | (n = 11) |
| Age (years) | Mean ± SE | 67.2 ± 1.3 |
| Gender: | | |
| Male | n (%) | 9 (81.8) |
| Female | n (%) | 2 (18.2%) |
| Weight (kg) | Mean ± SE | 84.6 ± 3.0 |
| Height (m) | Mean ± SE | 1.76 ± 0.03 |
| BMI (kg/m²) | Mean ± SE | 27.2 ± 0.8 |
| Duration of diabetes | Mean ± SE | 6.6 ± 1.4 |
| (years) | | |
| Class of anti-diabetic | | |
| medication: | | |
| Metformin (M) | n (%) | 3 (27.3) |
| Sulfonylureas (S) | n (%) | 5 (45.5) |
| Combination of M, S | n (%) | 3 (27.3) |
| HbA1c (%) | Mean ± SE | 6.85 ± 0.21 |
| Fasting glucose^{#} | Mean ± SE | 8.41 ± 0.33 |
| (mmol/L) | | |
| Fasting insulin (mU/l) | Mean ± SE | 6.87 ± 1.47 |
| Fasting glucagon | Mean ± SE | 15.51 ± 2.32 |
| (pmol/l) | | |
| Fasting triglycerides | Mean ± SE | 1.77 ± 0.17 |
| (mmol/l) | | |
| Fasting FFA (mmol/l) | Mean ± SE | 0.50 ± 0.04 |
| Fasting hs-CRP (mg/l) | Mean ± SE | 2.54 ± 0.63 |

^{#}Fasting glucose determined using CGMS

TABLE 3
Average amount of macronutrients/bolus

| | | Diabetes-specific | Standard |
|---|---|---|---|
| Ingredient | Unit | formula | formula |
| Energy (amount) | Mega Joule (MJ) | 1.65 | 1.65 |
| Protein | Gram | 19.3 | 14.9 |
| Carbohydrates | G | 45.6 | 53.4 |
| Fat | G | 14.9 | 13.4 |
| Fibers | G | 7.9 | 5.5 |

### Results

Twelve patients were included in the study. One patient terminated the study early and was excluded from data analysis. Table 2 shows the subject characteristics at baseline. The mean calculated energy requirements were 1967±237 kcal/day (minimum 1431 and maximum 2317 kcal), which corresponds to 393±47 kcal (and 393±47 ml) per bolus. The average amount of macronutrients provided per bolus is shown in Table 3.

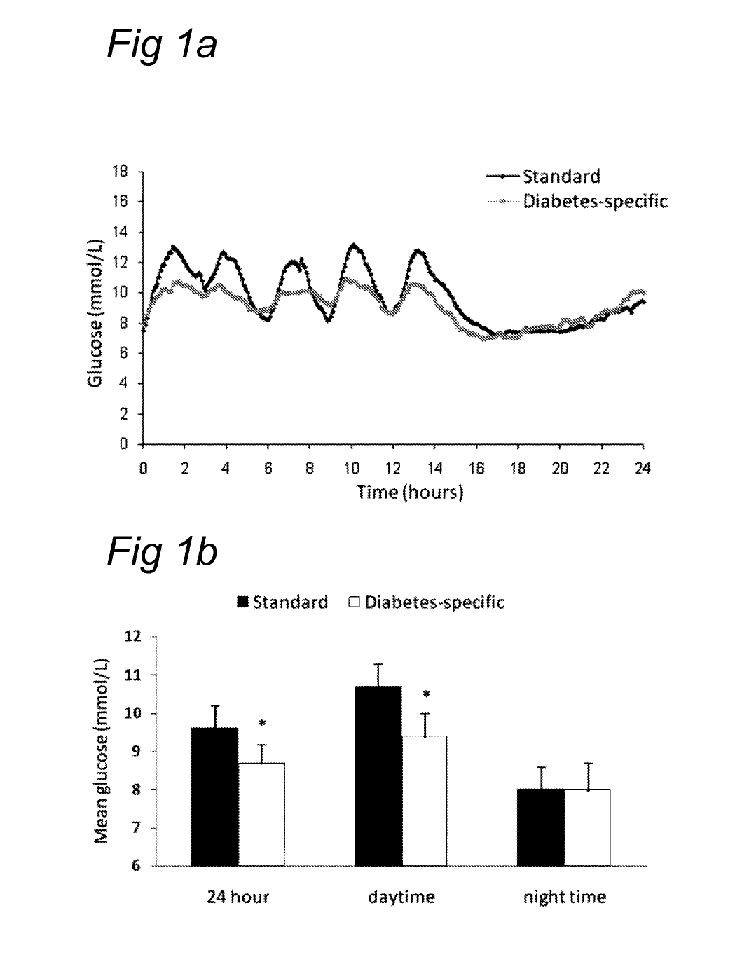

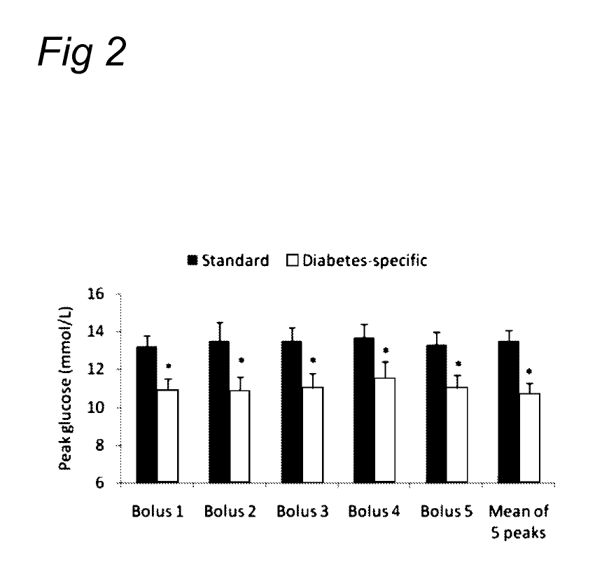

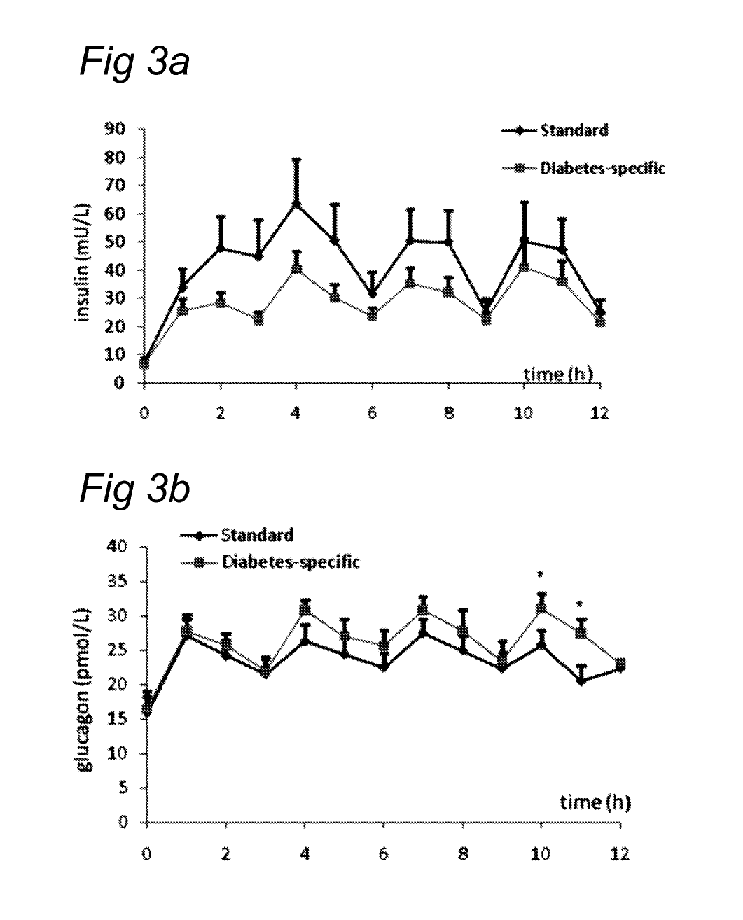

Fasting glucose levels at the initiation of the 24 h assessment were not significantly different between both formulas (7.6±0.37 mmol/L for the standard formula group and 7.9±0.45 mmol/L for the diabetes-specific formula group, p=0.61). FIG. 1A shows the 24 h glucose concentrations after bolus administration of both formulas. Mean glucose concentration was significantly lower after administration of the diabetes-specific formula compared with the standard formula during 24 h and daytime (FIG. 1B). No significant differences in mean glucose levels were observed at night time. Analysis of the 24 h glucose profile, measured as iAUC, also showed lower glucose levels during daytime with the diabetes-specific formula as compared with the standard formula (data not shown). However, the difference in iAUC was not statistically significant over 24 h (p=0.136) or during night time. Furthermore, administration of the diabetes-specific formula resulted in a 26% reduction of total hyperglycaemic time (>10 mmol/L) over 24 h compared with the standard formula (7.5±2.3 h versus 10.2±2.0 h, p<0.05). At daytime, the duration of hyperglycaemic episodes was 30% shorter than with the standard formula (6.2±1.6 versus 8.8±1.2 h; p<0.05). Hypoglycaemic episodes (<3.9 mmol/L) were hardly experienced during the 24 h assessments (<0.5% of the time) and no significant difference was observed between both formulas. The reproducibility of glucose measurements was checked by comparing five paired data points between the CGMS readings and the capillary blood finger prick tests and they were highly correlated, with a mean Pearson correlation coefficient of 0.843±0.052 and a regression coefficient of 0.911±0.052. Postprandial glucose responses to the formulas were assessed by analysing the (delta) peak glucose concentrations after each bolus. Significantly lower peak (FIG. 2) and delta peak (data not shown) glucose levels for every bolus were observed after administration of the diabetes-specific formula as compared with the standard formula. The mean of the five peak (FIG. 2) and delta peak (data not shown) glucose levels were also significantly lower after use of the diabetes-specific formula. The insulin response during administration of the formulas did not significantly differ at any of the time points as shown in FIG. 3A. The 24 h insulin value was also not significantly different between both study products. When the insulin response was assessed as iAUC (during first 12 h of the assessment), the use of the diabetes-specific formula resulted in a significantly lower insulin response as compared with the standard formula (272.1±44.4 and 427.4±99.8 mU/L min, respectively). The glucagon concentration was significantly higher at 10 and 11 h after start of the first bolus during feeding with the diabetes-specific feed as compared with the standard formula (FIG. 3B). After 24 h, no significant differences in glucagon were observed. The iAUC for glucagon over the first 12 h was not significantly different between the formulas (121.9±16.7, diabetes-specific and 99.2±19.8 pmol/L min, standard, p=0.342). No significant differences were found in the mean score of all tolerance parameters. No significant differences in the number of subjects with one or more adverse events or in the number of adverse events per subject were found between formulas. The presence of GI related adverse events was equally distributed between groups (two in the diabetesspecific group (flatulence and diarrhoea) and two in the standard group (bloated feeling and diarrhoea)). One serious adverse event occurred (vasovagal collapse) during administration of the standard formula. This event was probably associated with the manipulation of the nasogastric tube during suction.

The study results show that a new diabetes-specific formula, administered as five boluses during the day, results in a significantly improved 24 h glucose profile. Insulin responses were also lower following administration of the new diabetes-specific formula compared with the standard, fibre-containing formula. The contribution of postprandial glucose levels to overall glycaemic control ranges from 70% in patients in the lowest HbA1c quintile (<7.3%) to 30% in patients in the highest quintile (>10.2%). Besides their effect on overall glycaemic control, lower postprandial glucose concentrations after ingestion of the diabetes-specific formula may decrease the risk of shortterm symptoms of hyperglycaemia, like increased thirst, dehydration, weight loss, blurred vision and fatigue. Postprandial metabolic derangements induce oxidative stress and endothelial dysfunctions and on the long term they are important cardiovascular disease risk factors. Improved glucose control by using a diabetes-specific formula instead of a standard formula might beneficially affect clinical outcome in diabetic patients in need of nutritional support. The present study results confirm that a low glycaemic load, by using a diabetes-specific formula, is able to decrease (postmeal) glucose concentrations, this despite a significant increase of postprandial glucagon concentrations in blood after administration of more than 2 ready to use servings of the product. Several properties or ingredients of the tested diabetes-specific formula have contributed, alone or in combination to the lower glucose profiles, as compared with the standard formula. The digestible carbohydrate fraction has a specific character, as well as the fiber and protein fraction. Tolerance and adverse effects were comparable between the groups. These results indicate that this new diabetes-specific formula may help to improve glycaemic control.

### Experiment 2

### Test of a Product in Older Diabetics

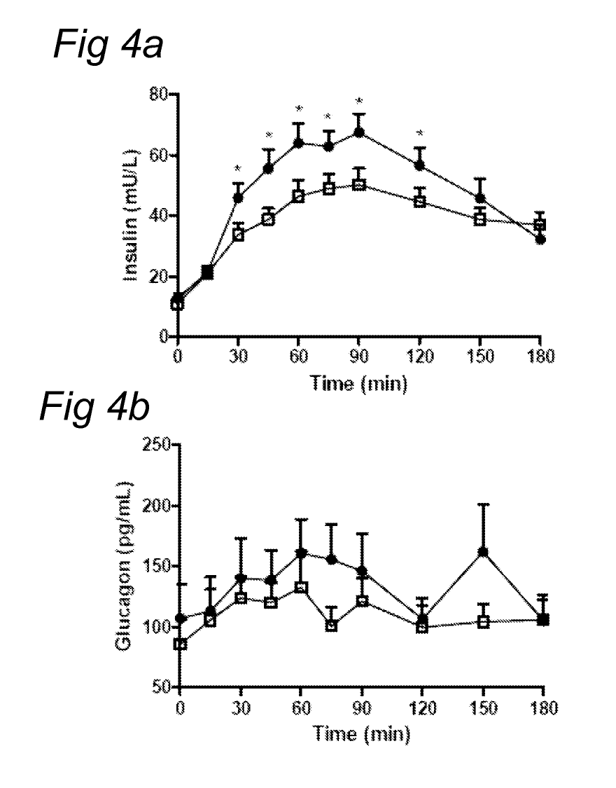

A group of 30 obese (average BMI 29±4) diabetes type II patients having a mean age of 63±7 yrs were randomized. They received a bolus of 75 g of product A (insulin, filled circles) or 75 g of product B (glucagon, open squares) in blinded way. Blood samples were drawn at 15, 30, 45, 60, 75 and 90 minutes and later every 30 minutes and insulin and glucagon measured. FIG. 4 demonstrate the differences found with respect to these parameters.

This experiment shows that the protein which was used (α-lactalbumin-enriched whey/soy 50/50) is capable of achieving the same pattern. In this study, the addition of the protein as claimed was compared with casein on a background matrix of a digestible carbohydrate. This digestible carbohydrate can be isomaltulose or glucose. It appeared that the protein fraction comprising the protein from vegetable origin demonstrated a similar postprandial glucose response but a significantly higher postprandial glucagon release, compared to the casein-based formula.

### Example 3

### Products

### Product 1

Composition comprising per 100 g dry matter more than 70 g of a protein fraction comprising 36 to 90 weight % protein of vegetable origin and 10 to 64 weight % of protein of dairy origin and optionally 0.2 to 6 g each of L-arginine, ornithine, carnosine or anserine. The product can be dry or be liquid.

### Product 2

Composition comprising per 100 g dry matter more than 80 g of a carbohydrate fraction which comprises 15 to 70 weight % galactose or lactose and 10 to 60 weight % isomaltulose. The composition can be liquid semi liquid or dry.

### Product 3

Ready-to-drink liquid nutritional product comprising 18 to 20 En % protein, 30 to 47 En % carbohydrates and 31 to 52 En % lipids, and further comprising per 100 ml:
4.5 to 5.2 g protein equivalents (Kjeldahl nitrogen times 6.25), whereby the protein comprises 40 to 60 weight % soy protein isolate and 40 to 60 weight % whey protein;
11.4 to 12 g digestible carbohydrates, wherein these carbohydrates comprise 3.2 to 4 g lactose, 4 to 4.9 g isomaltulose, 0.1 to 0.7 g glucose and 2.4 to 3.5 g polysaccharides as are present in commercially available resistant dextrin or resistant starch or tapioca starch;
3.3 to 4.3 g lipids of which 0.08 to 2 g is marine oil and the remainder originates from canola oil and sunflower oil;
1.6 to 2.4 g fiber of which 0.2 to 0.5 is insoluble and 1.1 to 2.2 is soluble, the fiber comprising cellulose;
35-140 mg choline;
vitamins, trace elements and minerals are included in amounts which fall within the current regulations for “food for special medical purposes”.

### Product 4

A nutritional product comprising per daily dose of 45 g dry matter:

1.2 g carnosine or anserine, 1.9 g betaine, 1.2 g melatonin and 8.4 g medium chain triglycerides which comprise more than 40 weight % octanoic acid.

1-16. (canceled)

17. A method for treating a disorder mediated by a postprandial endocrine response in a human body involving the sequential or simultaneous release of insulin and glucagon, the method comprising administering to a human a nutritional composition comprising:
(a) a protein fraction comprising at least 30 weight % of a vegetable protein,
(b) a digestible carbohydrate fraction comprising:
(i) 15 to 70 weight % of at least one of galactose and lactose, and
(ii) 10 to 65 weight % of isomaltulose, and
(c) a nutritional fiber fraction comprising 60 to 92 weight % of a soluble fiber.

18. The method according to claim 17, wherein the disorder is a gut motility disorder.

19. The method according to claim 18, wherein the gut motility disorder is selected from the group consisting of irritable bowel syndrome and gut cramps.

20. The method according to claim 17, wherein the composition further comprises one or more of melatonine, carnosine, anserine, octanoic acid choline, betaine and functional equivalents thereof.

21. The method according to claim 20, wherein the composition comprises one or more of carnosine, anserine, octanoic acid, and functional equivalents thereof.

22. The method according to claim 17, wherein the postprandial endocrine or neurological response is selected from at least 3 of the following group of responses: the release of insulin, glucagon, GLP-1, GLP-2, gastrin, amylin, PYY, GIP, reeelin, CCK, glicentin, hGH, IGF-1, oxytocin, vasopressin and melanocortins.

23. The method according to claim 17, wherein the digestible carbohydrate fraction comprise more than 40 wt % of the sum of (i) galactose or lactose and (ii) isomaltulose.

24. The method according to claim 17, wherein the protein fraction provides more than 16 en % of the total caloric content of the composition.

25. The method according to claim 17, wherein the composition has an energy density of more than 1 kcal/ml and comprises:
(i) less than 40 wt % casein,
(ii) less than 60 wt % carbohydrates having a glycemic index above 80, and
(iii) more than 30 en % of lipids and.

26. The method according to claim 17, wherein the protein fraction consists essentially of whey, optionally enriched in alpha-lactalbumin and soy in a whey:soy-weight ratio of 30:70 to 90:10.

27. The method according to claim 17, wherein the disorder is selected from the group of diabetes type II, diabetes mellitus, diabetes type I, metabolic syndrome, obesity (BMI>28), diabetes as a result from the use of atypical antipsychotics, hepatosteatosis, excessive food- or energy consumption, abnormal urinary losses of glucose, muscle atrophy, lipid accumulation in muscle tissue, neurological disorders, irritable bowel syndrome, gut cramps, stomach disorders, like gastric reflux, excessive acid production in the stomach, cardiovascular problems, undesired weight loss, insufficient body-temperature homeostasis, sleeping problems and DPP IV-mediated disorders.

28. The method according to claim 27, wherein the neurological disorder is selected from the group of age-related cognitive impairment, dementia, Alzheimer's, confusion, pathological thought disorders, schizophrenia, symptoms of schizophrenia, psychosis, affect disorders, depression, stroke, amyloid lateral syndrome (ALS), white matter abnormalities, nerve injury including spinal chord injury, multiple sclerosis (MS), cerebrovascular accidents (CVA), transient ischaemic accident (TIA), Parkinson's Disease (PD) and epilepsy.

29. The method according to claim 17, wherein the postprandial endocrine response is a neuro-endocrine response or comprises an interaction with a nervous system or the pituitary gland.

30. The method according to claim 29, wherein the interaction with a nervous system comprises stimulation of the nervus vagus, a change in the expression of serotonin receptors, an increased release of serotonine or an interaction with gaba-ergic neurons of the enteral nervous system, or an interaction with the hypothalamus, hippocampus, cerebellum, brain stem or pituitary gland.

31. The method according to claim 17, wherein administration of the composition delays gastric emptying, decreases acid release in the stomach, decreases urinary excretion of glucose and/or prevents a decrease of body temperature.

32. The method according to claim 17, wherein the vegetable protein comprises 6.5 to 9.5 weight % of L-arginine.

33. A method for treating a disorder mediated by a postprandial endocrine or neurological response in a human body selected from the release of at least 2 of the group consisting of insulin, glucagon, GLP-1, GLP-2, gastrin, amylin, PYY, GIP, reeelin, CCK, glicentin, hGH, IGF-1, oxytocin, vasopressin and melanocortins, the method comprising administering to a human a nutritional composition comprising:
(a) a protein fraction comprising at least 30 weight % of a vegetable protein,
(b) a digestible carbohydrate fraction comprising:
(i) 15 to 70 weight % of at least one of galactose and lactose, and
(ii) 10 to 65 weight % of isomaltulose, and
(c) a nutritional fiber fraction comprising 60 to 92 weight % of a soluble fiber.

34. The method according to claim 33, wherein the disorder is a gut motility disorder.

35. A method for treating a disorder mediated by a postprandial endocrine or neurological response in a human body, the method comprising administering to a human a nutritional composition comprising at least one of:
(a) a protein fraction comprising at least 30 weight % of a vegetable protein,
(b) a digestible carbohydrate fraction comprising:
(i) 15 to 70 weight % of at least one of galactose and lactose, and
(ii) 10 to 65 weight % of isomaltulose, and/or
(c) a nutritional fiber fraction comprising 60 to 92 weight % of a soluble fiber, wherein the composition further comprises one or more of melatonine, carnosine, anserine, octanoic acid, choline, betaine, and functional equivalents thereof.

36. The method according to claim 35, wherein the disorder is a gut motility disorder.
